(19) United States
(12) Patent Application Publication
(10) Pub. No.: US 2011/0313435 A1
(43) Pub. Date: Dec. 22, 2011

(54) SUTURE BUCKLE WITH SELECTIVE FRICTION

(75) Inventors: David Trottingwolf Aldridge, Laguna Hills, CA (US); Seth Arnold Foerster, San Clemente, CA (US)

(73) Assignee: DALLEN MEDICAL, INC., San Clemente, CA (US)

(21) Appl. No.: 13/161,833

(22) Filed: Jun. 16, 2011

Related U.S. Application Data
(60) Provisional application No. 61/355,456, filed on Jun. 16, 2010.

(51) Int. Cl. A61B 17/03

(52) U.S. Cl. 606/151

(57) ABSTRACT

A tensioning device for holding separated members/tissues in contact with one another. The device comprises a frame having opposing first and second sides and a lower surface and an upper surface, and a band for extending around the separated tissues to be held together in conjunction with the frame. The band has two ends releasably attached to the sides of the frame. When the ends are secured to the sides of the frame, the band establishes a path of tension along its length that extends linearly between the two ends of the band.

This application claims the benefit under 35 U.S.C. 119(e) of the filing date of Provisional U.S. Application Ser. No. 61/355,456, entitled Suture Buckle with Selective Friction, filed on Jun. 16, 2010, and expressly incorporated herein by reference, in its entirety.

This application is also related to commonly assigned U.S. patent application Ser. No. 12/815,989, entitled Suture Band Buckle and Methods, filed on Jun. 15, 2010, U.S. patent application Ser. No. 12/836,000, entitled Flat Suture Banding System and Methods, filed on Jul. 14, 2010, and U.S. patent application Ser. No. 12/858,332, entitled Low Friction Buckle Tightening System and Methods, filed on Aug. 17, 2010. Each of these three patent applications are herein expressly incorporated by reference, in their entirety.

### BACKGROUND OF THE INVENTION

The present invention is related to the general surgical repair of separated body tissues, and more particularly to internally fixating and stabilizing such body tissues, specifically bones.

In the present state of the art, there are a number of systems available to repair biological tissues separated in surgery or by injury. These products serve to approximate and stabilize the tissues so that healing may commence and provide compression in the interface to promote healing. Compression and stability are critical for proper anatomical healing of tissue. With the correct amount of compression applied to the interface of the tissue portions to be joined, signals are sent to the tissue, thus allowing the tissue to remodel in proper anatomical position. The amount of compression applied to the tissue interface needs to be appropriate to the type of tissue that is being healed.

Twisted wires are also typically used to keep bone fragments together so they may heal. Twisted wires only hold tension as long as the twisted wire pair remains stable. Often the wires untwist too soon failing to keep the bone fragments together so that they may heal. Wires can also cut into the bone fragments allowing them to separate so that healing is difficult.

When it is necessary to access the thoracic cavity for a medical procedure, for example, it is required to cut the sternum into two pieces using a sternal saw. Once the procedure is completed within the thoracic cavity, the sternum must be repaired. For such repairs, it is known to use a dynamic compression device. Some of the drawbacks of this typical device, and others which are used include:
1. Bulky spring materials, while occupying substantial space, often do not store much energy. Some use polymer elastic bands, while other use coiled springs;
2. Wires are sometimes used to wrap the bones into position in compression with one another. However, wires can have sharp ends that can damage adjunctive tissues. Knot stacks in suture can interfere with the natural movement of surrounding tissues; and
3. Current banding systems that incorporate a biasing mechanism to achieve dynamic compression put the biasing mechanism in line with the band or suture. This practice competes with precious space at the healing site. Suture or bands are used to approximate tissues so that they may heal. It is desirable to obtain the best purchase possible on the tissue, so that the binding mechanics offered by the suture may be utilized. The best purchase is optimized by ensuring that the suture has the greatest contact area with the tissue. If a biasing mechanism is interfering with this concept, the biasing mechanism may diminish the suture's ability to hold the tissues together.

In addition, the current banding systems have stiff bands that are not compliant with bony undulations. Flat sutures are used, but are tedious to tie and do not hold reliably.

The banding systems of the present invention are therefore attractive for use in sternal closure because they offer some distinct advantages over the twisted wires most commonly used in the procedure.

Bands address the issues wires have in the following discussion. A band, by definition, is wide. In being wide, a band distributes its forces over a wider surface area. This inhibits the band from digging into the bone. In being wide, a band affords a larger cross-sectional area whereby more material may be realized thus presenting the opportunity to offer as much strength in the construct as is necessary to hold the bone fragments together. As such, bands address wire's two main weaknesses, namely, digging into the bone fragments being held together and, not having sufficient cross sectional area.

Bands bring in other attributes other than strength and reduced pressure on the bone. Some of these attributes are difficult to manage. With strength comes stiffness, as mentioned elsewhere herein. The larger cross-section of the band significantly increases the stiffness of the band. While stiffness and rigidity are good attributes in that they can stabilize the bone union, these attributes can also prevent the band from following the contours of the bone when inserted. This can lead to capturing tissues underneath the band that ultimately destabilize the union as the tissues continue to compress and disappear over time.

Binding the band ends together can also impose some problems. Generally this involves a mechanism on one band end that interfaces with holes or slots or contours on the other band end. This creates a tensioning system that is incremental in nature. As in the twisted wire system, this mechanical interface of the two ends is the weakest link in the system. This mechanical interface becomes stronger as the incremental steps become larger. But larger incremental steps aren't conducive to fine tuning the tension, so this is problematic. Flat sutures have been used to tie tissues together but the residual tension supplied in such a knotted structure is insufficient for optimum healing. There is a lot of fuss/time associated with trying to keep and hold a desirable tension with these flat sutures. What is needed is an attachment means that provides variable tensioning.

Another problem associated with all banding systems is that their tension holding capabilities are not sufficient for the environment in which they operate. Tension holding ability can be increased or enhanced by increasing friction at the binding interface of the band. What is needed however is a banding system with the ability to hold tension by selectively increasing friction at the binding interface during locking and/or after locking without increasing friction while tensioning.

What is needed, therefore, are improved devices and techniques for holding two tissue portions in a state of compression and tension that address and overcome these shortcomings in an innovative way.

### SUMMARY OF THE INVENTION

The present invention pertains to a tensioning device for holding separated tissues in contact with one another. The invention is applicable to other non-surgical environments as well, basically applying to any setting wherein it is desired to clamp two separated members together using a buckle arrangement.

More particularly, there is provided a tensioning device for holding separated tissues in contact with one another, which comprises a frame. The frame has opposing first and second sides, a lower surface, and an upper surface, wherein the lower surface comprises a comb/serrated member comprising teeth on the second side. A band is provided for extending around the separated tissues to be held together in conjunction with said frame. The band has a first end attached to the first side of the frame and a second end that is securable to the second side of the frame, wherein, when the second end is tensioned along the second side of the frame, the band establishes a path of tension along its length that extends linearly between the two ends of the band.

The comb may be angled, in certain embodiments, to allow the second end of the band to pass over it without engaging the band while the band is tensioned. The teeth of the comb are big enough to penetrate the band, in order to mechanically engage the band at the second end. The comb is adapted to hold the tension using its teeth, by mechanically engaging or piercing the band at the second band end.

A lock bar is moveably attached to the second side of the frame via a bar attachment, wherein the lock bar and attachment are adapted to hold the tension by pinching the second band end between the bar and second frame side. The bar attachment is adapted to mechanically constrain movement of the lock bar into the inside of the frame and away from the upper surface about the second side of the frame. When the lock bar and bar attachment fail to hold the tension, then the teeth of the comb are adapted to fully pierce and engage the band at the second end and maintain the tension. In certain embodiments, the bar is attached inside the frame at the second side via a bar attachment that is adapted to mechanically constrain movement of the bar into the inside of the frame and away from the upper surface about the second side of frame.

The space between the lock bar and the inside of the first side of the frame, and the space between the bar and the inside of the second side are preferably substantially the same size, and are sized to allow the band to pass through easily. The lock bar preferably has a circular cross section and the bar attachment is adapted to constrain the bar from rotational movement.

In certain embodiments, the lock bar is attached outside the second side of the frame via a bar attachment that is adapted to mechanically constrain movement of the lock bar into the inside of the frame and away from the upper surface about the second side of the frame.

A needle may be attached to the second end of the band. The frame has an opening sized to allow passage of the needle through it. The lock bar is adapted to move away from the second frame side to a sufficient distance so as to allow the needle to pass around the lock bar. The inventive device may be adapted to be used in conjunction with a needle guide, wherein the needle guide comprises integral slots and is sized to removably fit, via the slots, over the lockbar in order to restrain the lock bar, and the needle guide is adapted to mitigate or prevent damage from the needle to the lock bar and bar attachment when the needle guide is placed over the lock bar. The second band end is routed around and through tissues being held together and from the bottom up through the center of the frame and over the bar and down between the lock bar and the second side of frame, such that when the second band end is tensioned and released, the bar holds the tension by pinching the band between the lock bar and the second frame side, and the comb further holds the tension via the teeth by mechanically engaging or piercing the band at the second band end. The band may be substantially flat.

In another aspect of the invention, there is provided a surgical tensioning device for holding separated tissues in contact with one another, which comprises a frame. The frame has opposing first and second sides, a lower surface, and an upper surface, as well as a band for extending around the separated tissues to be held together in conjunction with the frame. The band has a first end that is attached to the first side of the frame, and a second end that is releasably securable to the second side of the frame, wherein, when the second end is secured to the second sides of the frame, the band establishes a path of tension along its length and extends linearly between the two ends of the band. A lock bar is movably attached parallel to the second side of the frame via a bar attachment. The lock bar and attachment are adapted to hold the tension by pinching the second band end between the lock bar and the second frame side. The lower surface of the frame comprises a comb comprising teeth on the second side, the comb adapted to further hold the tension via the teeth by mechanically engaging or piercing the band at the second band end.

In yet another aspect of the invention, there is provided a tensioning device for holding separated tissues in contact with one another which comprises an elongate member and a frame having first and second side, a lower surface, and an upper surface. The elongate member, preferably a flat band, has a first end that is attachable to the first side of the frame and a second end that is releasably securable to the second side of said frame. A movable clamping member on the frame is adapted to secure the second end of the elongate member to the second side of the frame by cinching the second end of the elongate member between an engagement surface on the elongate member and a mating engagement surface on the second side of the frame. A comb/serrated member is disposed on the lower surface of the second side of the frame. Accordingly, when the elongate member is tensioned to the predetermined level and is secured to the second side of the frame, the elongate member establishes a path of tension along its length that extends linearly between the two ends of the elongate member.

In a preferred embodiment, the comb is angled to allow the second end of the elongate member to pass over it without engaging the elongate member while the elongate member is secured to the second side of the frame. The teeth of the comb are big enough to penetrate the elongate member to mechanically engage the elongate member at the second end. The comb is adapted to hold the tension using its teeth by mechanically engaging or piercing the elongate member at the second end of the elongate member. The movable clamping member comprises a lock bar moveably attached to the second side of the frame via a bar attachment, wherein the lock bar and attachment are adapted to hold tension by pinching the elongate member at the second end between the bar and second frame side.

Preferably, the lock bar has a generally circular cross section and the bar attachment is adapted to constrain the bar from rotational movement. The bar attachment is adapted to mechanically constrain movement of the lock bar into the inside of the frame and away from the upper surface about the second side of the frame. When the lock bar and bar attachment fail to hold the tension on their own, then the teeth of the comb are adapted to fully pierce and engage the elongate member at the second end and maintain the desired tension.

The bar is attached inside the frame at the second side via a bar attachment that is adapted to mechanically constrain movement of the bar into the inside of the frame and away from the upper surface about the second side of frame. The lock bar is attached outside the second side of the frame via a bar attachment that is adapted to mechanically constrain movement of the lock bar into the inside of the frame and away from the upper surface about the second side of the frame.

In preferred embodiments, a needle is attached to the second end of the elongate member. The frame has an opening sized to allow passage of the needle through it. A needle is also attached to the second end of the elongate member, wherein the lock bar is adapted to move away from the second frame side to a sufficient distance so as to allow the needle to pass around the lock bar.

The foregoing described device is adapted to be used in conjunction with a needle guide, wherein the needle guide comprises integral slots and is sized to removably fit, via the slots, over the lockbar in order to restrain the lock bar. The needle guide is adapted to mitigate or prevent damage from the needle to the lock bar and bar attachment when the needle guide is placed over the lock bar.

The invention, together with additional features and advantages thereof, may best be understood by reference to the following description taken in conjunction with the accompanying illustrative drawings.

### BRIEF DESCRIPTION OF THE DRAWINGS

FIG. 1A is a top view of a common buckle;

FIG. 1B is a cross sectional view of the buckle shown in FIG. 1a, fitted with a strap;

FIG. 2 is a top view showing how wires and bands might be used in binding sternal halves together for healing after open heart surgery;

FIG. 3A is a perspective view of a portion of suture band buckle according to one embodiment of the invention, wherein the fixed suture at one end of the buckle is shown;

FIG. 3B is a perspective view of the suture band buckle shown in FIG. 3A with the suture at the both ends of the buckle shown;

FIG. 3C is a cross sectional view of the buckle in FIG. 3A into which the loose end of suture has been secured;

FIG. 4A is a perspective view of a portion of suture band buckle wherein the fixed suture at one end of the buckle is shown, according to another embodiment of the invention;

FIG. 4B is a perspective view of the suture band buckle shown in FIG. 4A with the suture at the both ends of the buckle shown;

FIG. 4C is a cross sectional view of the buckle of FIGS. 4A and 4B, into which the loose end of suture has been secured;

FIG. 5 is a perspective view of a suture band buckle, with suture and a needle attached to the suture and, a needle; FIG. 7A shows the needle being guided by the needle guide into the buckle; FIG. 7B shows the suture and needle being guided out of the buckle;

FIG. 6 is an isometric view of a suture band buckle, into which two loose ends of suture have been secured;

FIG. 7A is an enlarged view of the bottom portion of a suture band buckle in accordance with the present invention; FIG. 7B is an enlarged view of the bottom portion of the suture band buckle in FIG. 7A wherein a portion of the suture at one end is shown and is secured;

FIG. 8 is an enlarged view of comb/serrated member present in an embodiment of the present invention including the embodiment in FIG. 7, shown as engaging a portion of suture;

FIG. 9 is a perspective view of a suture band buckle in accordance with an embodiment of the present invention shown in FIG. 7, with a needle guide and into which both ends of the suture have been secured.

### DESCRIPTION OF THE PREFERRED EMBODIMENTS

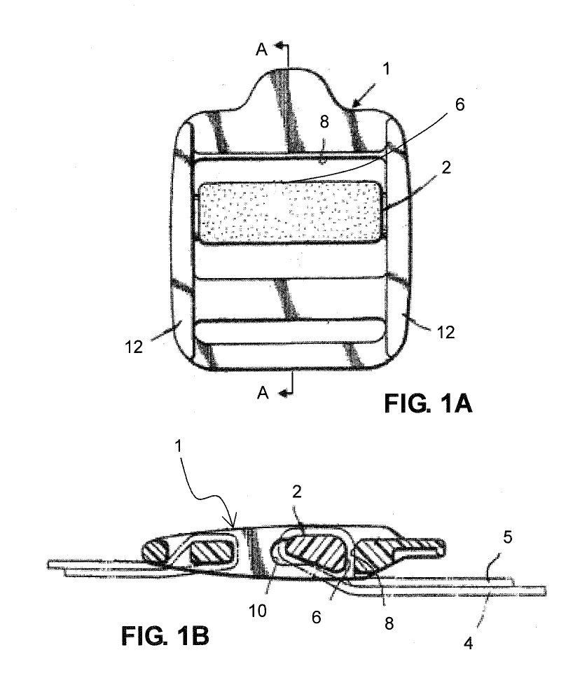

Referring to FIGS. 1A and 1B, a common buckle 1, used in straps on bags, for example, might be considered in this application for its variable tensioning abilities. A lock bar 2 moves a surface 6 away from a surface 8 when a strap tail 5 is tensioned. As the strap tail 5 is tensioned, another strap tail 4 is pulled into the 6/8 surface interface. When strap tail 5 is released, the strap tail 4 holds the dominant tension which pulls the lock bar 2 so that the strap is pinched between surfaces 6 and 8. This is the locked position of the buckle 1. Locking bar 2 is held in position by tabs on both of its ends, which ride in a slot 10. The slot 10 is formed in a frame 12. Slot 10 is important, because it holds the face 6 on the locking bar 2 so that it correctly pinches the strap up against the surface 8. The variability of the tension in each of the strap tails 4 and 5, relative to each other, cause the lock bar to move in slot 10. The lock bar naturally wants to even out these tensions by rotating in the slot 10. Flats are milled in the ends of the lock bar 2, to ride in the slot 10 so that such rotation does not happen.

This buckle generally has the means to provide variable tensioning to the strap loop. However with a suturing system, it is necessary to have one of these strap ends free so that it may be routed through tissue with a common suture-end-needle arrangement.

The common buckle 1 comes pre-threaded. What would be needed to make a buckle suitable for a suture application of the type contemplated in this application would be to have the buckle designed so that it might be threaded by the surgeon. In its common configuration, there is not enough room around the locking bar 2 to fit a suture needle.

The distance that the locking bar 2 has to travel to achieve this locking and unlocking is rather small. So the slot 10, provided for locking bar ends, is not long. Should this be scaled down to the size of a 5 mm wide suture band, the slot only needs to be 0.020 inches long. At this size, the slot does not provide bar travel sufficient to allow a common needle, 0.065 inches in diameter, to be threaded through the buckle. Furthermore, it would be difficult to make this common buckle design open up enough to make such threading sufficiently easy during a surgical procedure. Surgeons would like to see an obvious opening for the needle to pass through.

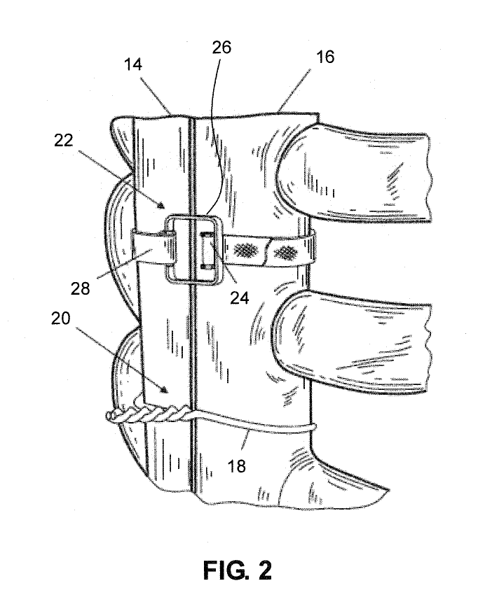

Pictured in FIG. 2 is a sternum that has been cut down the middle (osteotomy) for surgical access to the chest cavity. This osteotomy is commonly performed in open heart procedures that require surgical access to heart valves and heart arteries. The sternum is cut into two halves 14 and 16 and the chest is separated to gain access to internal organs. After surgery, closure of the sternum must be performed to ensure proper healing.

Shown in FIG. 2 are wires 18 that are commonly used to secure the sternal halves by wrapping them around the sternum and twisting them together at 20. Wires have many complications when used in this capacity. Wires 18 are thin and cut into the bone. The forces realized by twisting the wires are highly variable. The twisted wires can untwist when the patient coughs. The wires can cyclically fail, fall off the sternum, and migrate to puncture arteries and organs.

All of the failures of wires are addressed by the use of a buckled band system 22. FIG. 2 shows a tensioning system application for a single use buckled band system 22 employed after a sternal osteotomy procedure. The buckled band system 22 comprises a buckle 24 with a frame 26 and a band 28 that is wrapped around sternal halves 14 and 16 and terminating into. The band 28 is tightened by cinching the band through the buckle 24. Buckled band 22 is ideal for the present invention as it is a single use buckle. Band 28 can be cinched through buckle 24 in a reduced friction environment, until a programmed force triggers a mechanism within the buckle and the buckle locks down onto the band.

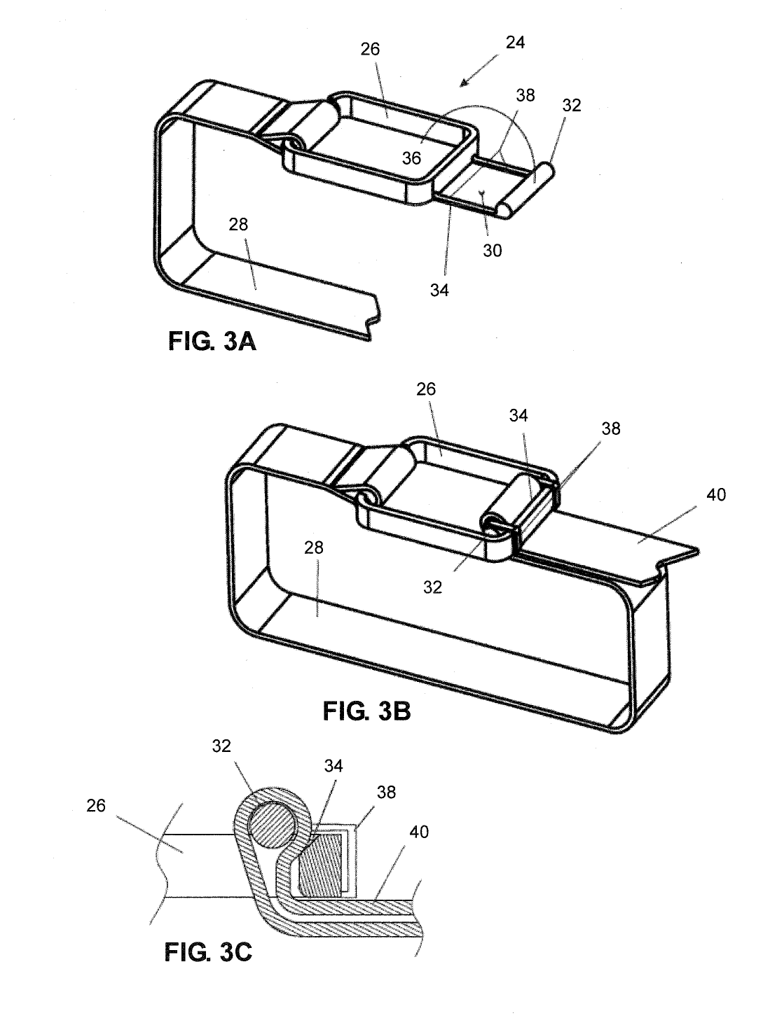

FIGS. 3A-3C represent a suture band buckle 24 that is a variant of the buckled system 22 of FIG. 2. As shown in FIGS. 3A and 3B, the buckle 24 comprises the frame 26 with a member 32 linked via attachments 38 to frame 26. The member 32 is in the form of a pin or bar in this embodiment. In addition, the member 32 may be rigid. Attachments 38 are in the form of straps in this embodiment. These attachment members that link the rigid pin or bar 32 to the frame 26, are preferably constructed to yield to slight pressure and form around frame 26. Pin or bar 32 ultimately pinches the suture up against the inside surface 34 of frame 26. One tail of suture or band 28, the first end, comes attached to the frame in this embodiment. The other tail or second end of the suture or band 28 is easily threaded into the buckle 24 through a window 30 held open by the attachment straps 38. As tension is applied to a suture tail 40, the pin or bar 32 rotates in a direction shown by an arrow 36 to go from the outside of frame 26 to the inside of frame 26. Attachment straps 38 yield and form around frame 26 in this process. The straps serve to hold the pin in proper position to pinch the suture against inside surface 34 when tension is released on the suture tail 40. Straps 38 also keep the bar or pin 32 from rotating. Although round bars or pins engage flat surfaces in this embodiment, contoured pins with contoured mating surfaces on the pin and frame respectively, may instead be used to increase the buckle's holding performance. The mating surfaces, contoured or otherwise, may be further roughened or textured if more performance or better grip or holding is required.

Buckle 24 has the attachment straps 38 that perform the same function as slot 10 does in buckle 1, yet buckle 24 is simple enough to allow the free second end of the suture tail 40 to be threaded through the buckle. Attachment straps 38 provide the same two functions as slot 10 does in buckle 1; first to guide the pin into its mating locking inside surface 34, and second to keep the pin or bar 32 from rotating in the lock. Attachment straps 38 also hold a window open to allow the threading of free suture end 40. Although the buckles represented in FIG. 2 are capable of being threaded, it would be difficult to manage with gloved fingers on such a small scale. This is all done in a low profile device as is required for such surgical implants. A downsized version of buckle 1 would have a larger profile due to the material needed around the slots.

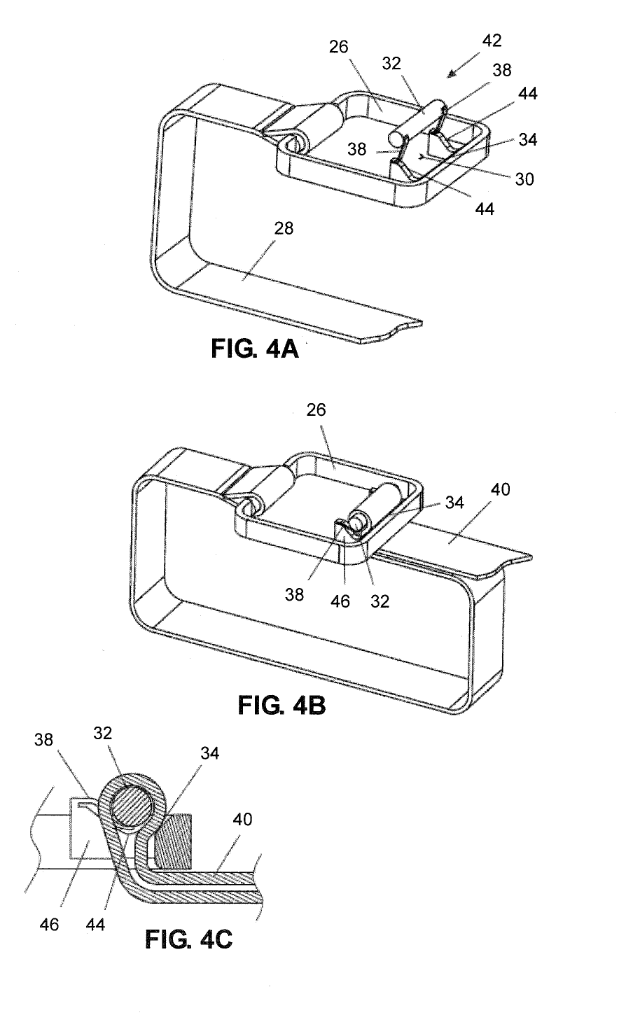

In FIGS. 4A-4C is illustrated a modified embodiment of the present invention. A buckle 42 is shown, which is similar to the buckle 24 in FIGS. 3A-3C in that it has the same frame 26 and bar or pin 32 and attachment straps 38. Straps 38, however, start in the interior of the frame instead of outside of the frame. This is managed by placing a buttress 46 on the interior of the frame and supplying a cam surface 44 to aid the straps in the guiding bar or pin 32. Consequently, the space or window 30 is supplied inside the frame for threading loose suture end 40. All the same advantages are realized with buckle 42 as buckle 24. Buckle 42 has the additional advantage of having a simple threading route whereas the threading route afforded by the buckle 24 is more complicated.

The cam surface 44 and strap 38 combination provide the same two functions that the slot 10 does in FIG. 1; first to guide the pin into its mating locking inside surface 34, and second to keep pin 32 from rotating in the lock. Cam surface 44 has the additional advantage of supplying more leverage to the hinging strap mechanism such that pin 32 is more forcefully pushed into and better locked inside the surface 34. Although round bars or pins are shown in this embodiment, contoured pins with contoured mating surfaces on the pin and frame, respectively, may be used to increase the buckle's holding performance. The mating surfaces, contoured or otherwise, may be further roughened or textured if more performance or better grip or holding is required.

The design criterion for these buckles is to present an easily threaded buckle to the physician. Thus far, this has been managed by opening up the buckle's architecture to allow for the suture to be threaded around pin 32. Due to the size of pin 32 and straps 38, the large needle needed for these procedures could easily push and bend pin 32 and straps 38 so they do not function optimally. What is needed is something that can both guide the needle around pin 32 and hold pin 32 so that the needle is not prone to disturb the functionality of the pin 32 and strap 38. Such a guide should facilitate the two passes the needle needs to make to route the suture 28 around the pin 32. The first pass comes up from the bottom and is the most difficult to manage as the needle is hard to see underneath the buckle. The second pass is easier to see as it originates from the top of buckle 42.

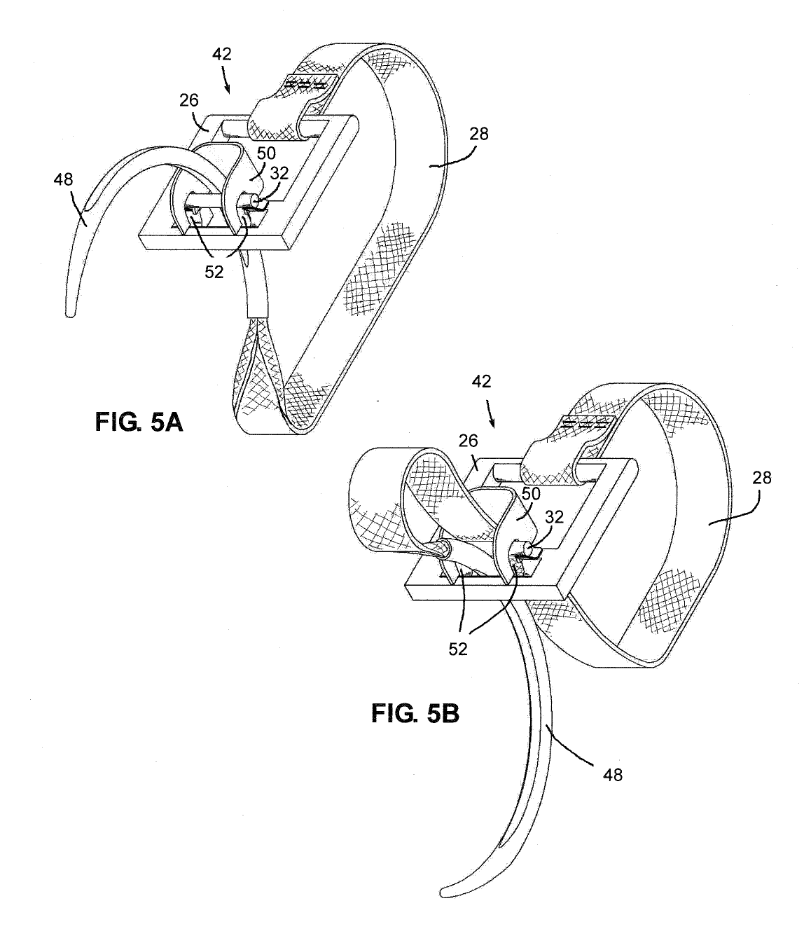

FIGS. 5A and 5B show a design development of the buckle 42 that presents a clear path within which the needle 48 is guided around pin 32 by means of a guide 50. FIG. 5A shows needle 48 in this path on the way up behind pin 32. The inner surface of the guide 50 serves to provide this path behind pin 32, which is the harder of the two needle passes to manage as the needle comes up blind, underneath buckle 42. The needle tip needs to find the interior of guide 50 and then can slide along this surface without disturbing pin 32. Should needle 48 accidently hit pin 32, slots 52 in the guide 50 restrain the movement of the pin 32, so that damage is not realized to pin 32.

FIG. 5B shows needle 48 on its way down in front of pin 32. In this configuration, visualization is much better. But still, there can be miscalculations in needle 48 movement that damage pin 32. Guide 50 mitigates or prevents this again with integral slots 52 which serve to restrain pin 32 such that damage to pin 32 or straps 38 is not realized.

Buckle 42 has been designed to be small because this is a desirable quality for orthopedic implants. Needle 48 needs to be abnormally large so that it can pierce sternal bone. Sternal needles are some of the largest needles made for threading sutures. While the guide 50 is effective in guiding needle 48 around the pin 32, it does not provide enough room to accommodate the large sternal needle 48. The present invention addresses this issue in the design of slots 52. Slots 52 in guide 50 are widened to allow lateral movement of pin 32 within the slot. This allows some of the space within guide 50 to be utilized for both the upward and downward passes of needle 48.

Guide 50 is a removable element releasably attached to frame 26. Once buckle 42 is threaded and locking pin 32 is set, guide 50 may be removed as it has no further function. This is desirable as this lowers the profile of the implant.

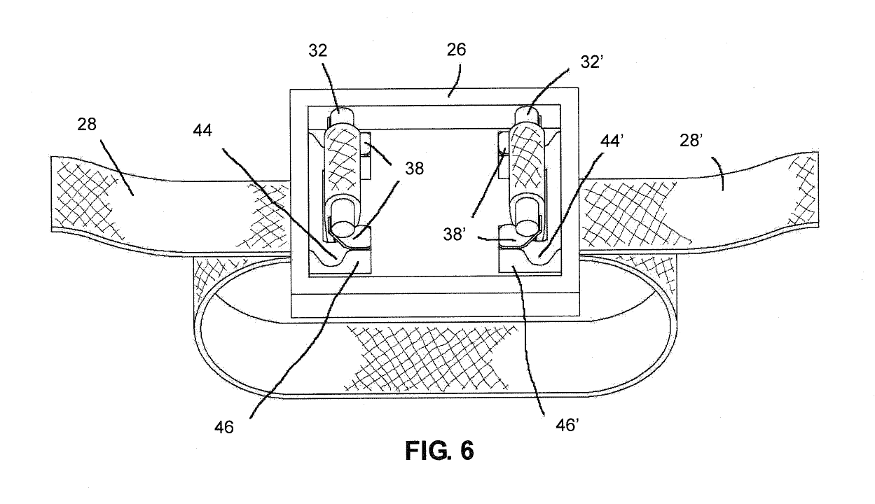

Further, it is also common, in surgical procedures, to work in tight spaces that require a small vertical access to the buckle. The present invention has one buckle on frame 26 with which to tighten the suture 28. When the tail or end of the suture 28 is pulled to tighten the repair, the frame 26 can be pulled off to the side. This is not acceptable, and frame 26 must be re-centered on the repair before tensioning proceeds. After centering frame 26, tensioning proceeds but immediately pulls frame 26 off to the side again. This pulling and centering becomes a tedious process that has an easy remedy. As a remedy, described herein is a buckle, shown in FIG. 6, that has two opposed sets of locking members in the form of bars or pins 32 and 32′ with related locking mechanisms using straps 38 and 38′ on frame 26 so that the two opposing suture tails or ends can be pulled in unison and secured by the respective bars or pins 32 and 32′ on frame 26, thus keeping the implant on center. As shown in FIG. 6, the bars or pins 32 and 32′ can be disposed inside the frame in a similar fashion as shown in FIGS. 4A-4C and 5A-5B, with buttresses 46 and 46′ on the interior of the frame and supplying cam surfaces 44 and 44′ to aid the straps 38 and 38′ in guiding bars or pins 32 and 32′. In an alternate buckle, the bars or pins 32 and 32′ can be disposed outside the frame in a similar fashion as shown in FIG. 3A-3C. The bars or pins 32 and 32′ and the attachments 38 and 38′, respectively, are preferably, substantially identical except that they are disposed on opposite sides of frame 26.

So far, buckles 24 and 42 that are both easy to thread, have good strength yet have a superior holding power, and are small so as to be used with suture sized straps, have been described. In miniaturizing the design, the buckles have been further modified with a needle guide to facilitate threading the buckle with larger needles. However, sometimes, the tension holding capabilities are not sufficient for the environment in which the buckles operate. An easy way to boost the buckle's tension holding abilities is to increase the friction in this binding interface. This may be done by altering the surface roughness at 34 or at pin 32. The problem, however, with altering the surface roughness is that it also adds friction to the initial tensioning step, making tensioning harder as well. Ideally therefore, the friction would have to switch passively off while tensioning and switch on while locking, as described below in the present invention.

Buckles are also used as fasteners in many industries. These buckles employ friction surfaces within their buckles' locking mechanisms to boost performance. The present invention is unique in that the buckle is used only once. In an inventive step of the present invention, because the buckle is used only once, common frictional surfaces are exchanged for interference mechanisms that are so aggressive that they may damage the suture. As can be seen in FIGS. 3A-3C, 4A-4C, and 6, the area where the suture tail 40 (or suture ends 28 and 28′ in FIG. 6) contacts frame 26 is an ideal place to insert such an interference mechanism. Suture tail 40 is carrying no load. Suture 28 is carrying the entire load on the other side of the lock. If some of this load can be placed on suture tail 40 with the aid of an interference mechanism, this will add to the buckle's holding abilities.

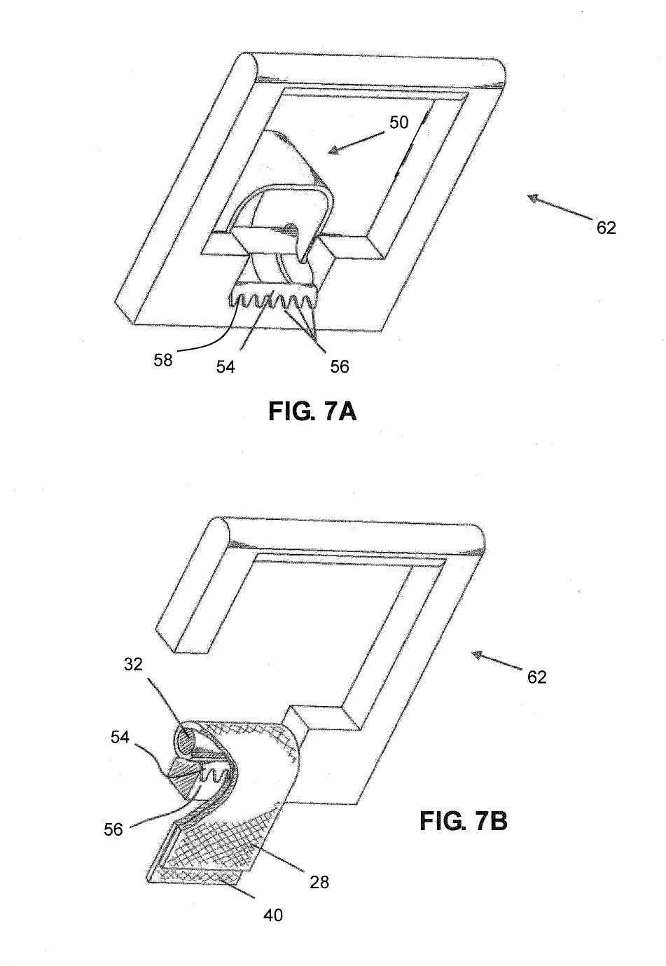

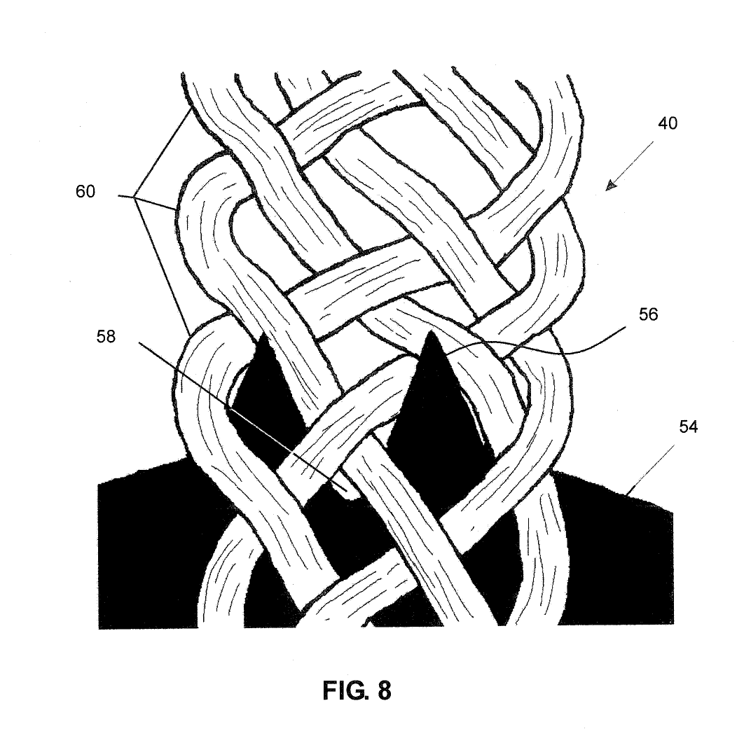

The present invention makes use of a comb or a serrated member to apply this aggressive and somewhat destructive hold on suture tail 40. As shown in an embodiment of the present invention in FIGS. 7A-7B, 8, and 9, and with particular reference to FIGS. 7A-7B and 8, a comb 54 with teeth 56 (and spaces or valleys 58 between the teeth 56) is used to apply this hold or to further hold the suture band end 40 in tension. Although the present embodiment is described and depicted in FIGS. 7A-9 with the comb 54 added onto a buckle such as the one shown in FIGS. 4A-4C, it will be readily apparent to a person of ordinary skill in the art can that a comb element 54 can be adapted for any buckle-suture system known in the art or described and shown herein. Teeth 50 are of a size and sharpness that can penetrate the braid on suture tail 40. While shown much bigger than yarns 60 in FIG. 8, teeth 56 need only be big enough to penetrate the yarns 60 to gain a mechanical purchase rather than a frictional purchase on suture tail 40. Valleys 58 between the teeth 56 may be smaller than the yarns 60 so that the yarns 60 may become jammed and stuck between adjacent teeth 56. Teeth 56 may also penetrate individual yarns 60 to gain sufficient purchase on suture tail 40.

Shown in FIGS. 7A and 7B is buckle a 62 constructed in accordance with another embodiment of the present invention, with a comb attached to its frame 26 substantially at the lower surface, where the suture tail 40 contacts the frame 26. Comb 54 is angled to allow the suture tail 40 to pass over teeth 54 with no engagement while suture is being tensioned.

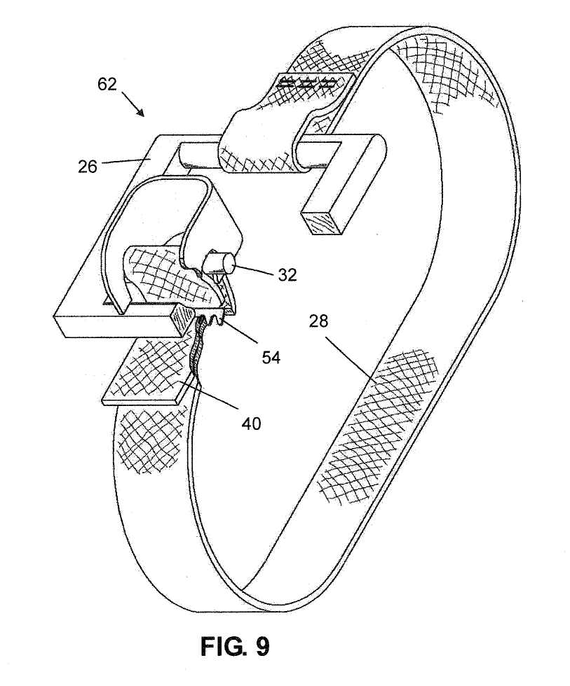

While a suture is being tensioned in the device of the present invention, after the desired tension is realized, suture tail 40 is released and is pressed onto teeth 56 on the comb 54 in the present invention as tension is shifted to suture 28. Both sutures will momentarily change directions of travel as the tension shifts and the lock bar settles in the lock. Comb 54 is angled so that teeth 50 preferably engage suture tail 40 after this shift in direction. If significant tension loads are subsequently put on suture 28, such that the locking mechanism in the buckle slips, teeth 56 will fully pierce and engage the suture tail 40. Valleys 58 in the comb 54 may cut, bind and hold yarn strands 60 within the suture tail 40. Also shown in FIG. 9 is a top view of the circlage buckle device 62 after it has been used, with a cutaway to expose placement of comb 54 within the buckle 62.

While the inventive concept is disclosed as being particularly adapted for use in repairing the sternum after a thoracic cavity procedure, it is, of course, applicable to a great many other procedures requiring repair of bodily tissue, particularly bone. In addition, the invention is applicable to other non-surgical environments as well, basically applying to any setting wherein it is desired to clamp two separated members together using a buckle arrangement.

The tissue portions to be held in contact with one another in the present invention comprise biological tissue in the body, including, but not limited to, skin, tendon, bone, ligaments, blood vessels, and organs. In one embodiment, the tissue portions comprise sternal bone that has been cut for a procedure to access the thoracic cavity. The suture may comprise woven, braided, or knitted fibers or metals, or a monofilament, and can be made of any known suture material. The suture may be of any shape, including, but not limited to, round, square, oval, flat (like a strap), or tubular. The shape of the suture for particular embodiments will be discussed more fully herein below. In one embodiment of the inventive device, the suture preferably comprises flat or tape suture.

In certain preferred embodiments, the buckle or frame of the device, as well as the pins or bars of the device, are fabricated from either tempered stainless steel, spring tempered stainless steel or titanium.

Although round bars or pins are shown up against flat surfaces in certain embodiment, contoured pins with contoured mating surfaces on the pin and frame respectively, may be used to increase the buckle's holding performance. The mating surfaces, contoured or otherwise, may be further roughened or textured if more performance or better grip or holding is required.

In certain preferred embodiments of the present invention, attachments or straps of the device are fabricated from either spring tempered stainless steel or titanium.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention, which is to be limited only in accordance with the following claims.

What is claimed is:

1. A tensioning device for holding separated tissues in contact with one another, the device comprising:
a frame, said frame having opposing first and second sides, a lower surface, and an upper surface, wherein said lower surface comprises a comb/serrated member comprising teeth on said second side; and
a band for extending around said separated tissues to be held together in conjunction with the frame,
said band having a first end attached to the first side of said frame and a second end that is securable to the second side of the frame, wherein, when the second end is tensioned along the second side of said frame, said band establishes a path of tension along its length that extends linearly between the two ends of the band.

2. The device of claim 1 wherein said comb is angled to allow the second end of said band to pass over it without engaging said band while the band is tensioned.

3. The device of claim 1 wherein the teeth of said comb are big enough to penetrate said band to mechanically engage the band at said second end.

4. The device of claim 1 wherein said comb is adapted to hold said tension using said teeth, by mechanically engaging or piercing the band at the second band end.

5. The device of claim 1, and further comprising a lock bar moveably attached to the second side of said frame via a bar attachment, said lock bar and attachment being adapted to hold said tension by pinching said second band end between said bar and second frame side.

6. The device of claim 5 wherein said bar attachment is adapted to mechanically constrain movement of said lock bar into the inside of said frame and away from said upper surface about the second side of the frame.

7. The device of claim 5 wherein when said lock bar and bar attachment fail to hold said tension, then the teeth of said comb are adapted to fully pierce and engage said band at said second end and maintain said tension.

8. The device of claim 5 wherein said bar is attached inside the frame at the second side via a bar attachment that is adapted to mechanically constrain movement of said bar into the inside of said frame and away from said upper surface about the second side of frame.

9. The device of claim 8 wherein the space between the lock bar and the inside of the first side of said frame and the space between the bar and the inside of the second side are substantially the same size, and are sized to allow said band to pass through easily.

10. The device of claim 8 wherein said lock bar has a circular cross section and said bar attachment is adapted to constrain said bar from rotational movement.

11. The device of claim 6 wherein said lock bar is attached outside the second side of said frame via a bar attachment that is adapted to mechanically constrain movement of said lock bar into the inside of said frame and away from said upper surface about the second side of the frame.

12. The device of claim 11 wherein said lock bar has a circular cross section and said bar attachment is adapted to constrain said bar from rotational movement.

13. The device of claim 1 and further comprising a needle attached to the second end of the band.

14. The device of claim 13 wherein said frame has an opening sized to allow passage of said needle through it.

15. The device of claim 9 further comprising a needle attached to the second end of the band wherein said lock bar is adapted to move away from said second frame side to a sufficient distance so as to allow the needle to pass around said lock bar.

16. The device of claim 15 adapted to be used in conjunction with a needle guide, wherein said needle guide comprises integral slots and is sized to removably fit, via said slots, over said lockbar in order to restrain said lock bar, and said needle guide is adapted to mitigate or prevent damage from said needle to said lock bar and bar attachment when said needle guide is placed over said lock bar.

17. The device of claim 8 wherein said second band end is routed around and through tissues being held together and from the bottom up through the center of the frame and over said bar and down between the lock bar and said second side of frame such that when the second band end is tensioned and released, said bar holds said tension by pinching said band between said lock bar and said second frame side and said comb further holds said tension via said teeth by mechanically engaging or piercing said band at said second band end.

18. A surgical tensioning device for holding separated tissues in contact with one another, the device comprising:
a frame, said frame having two opposing first and second side, a lower surface, and an upper surface;
a band for extending around said separated tissues to be held together in conjunction with said frame, said band having a first end that is attached to the first side of said frame and, a second end that is releasably securable to the second side of said frame, wherein, when the second end is secured to said second sides of said frame, said band establishes a path of tension along its length and extends linearly between the two ends of the band; and
a lock bar moveably attached parallel to the second side of said frame via a bar attachment said lock bar and attachment being adapted to hold said tension by pinching said second band end between said lock bar and said second frame side;
wherein said lower surface of said frame comprises a comb comprising teeth on said second side, said comb adapted to further hold said tension via said teeth by mechanically engaging or piercing said band at said second band end.

19. A tensioning device for holding separated tissues in contact with one another, the device comprising:
an elongate member;
a frame having first and second sides and a lower surface, and an upper surface, said elongate member having a first end that is attachable to the first side of said frame and a second end that is releasably securable to the second side of said frame;
a movable clamping member on said frame which is adapted to secure the second end of said elongate member to the second side of said frame by cinching the second end of said elongate member between an engagement surface on the elongate member and a mating engagement surface on said second side of the frame; and
a comb/serrated member on the lower surface of the second side of said frame,
wherein when said elongate member is tensioned to said predetermined level and is secured to the second side of said frame, said elongate member establishes a path of tension along its length that extends linearly between the two ends of the elongate member.

20. The device of claim 19, wherein said elongate member is a band.

21. The device of claim 19, wherein said comb is angled to allow said second end of said elongate member to pass over it without engaging said elongate member while said elongate member is secured to the second side of said frame.

22. The device of claim 19, wherein said teeth of said comb are big enough to penetrate said elongate member to mechanically engage said elongate member at second end.

23. The device of claim 19, wherein said comb is adapted to hold said tension using said teeth by mechanically engaging or piercing said elongate member at said second end of said elongate member.

24. The device of claim 19, said movable clamping member comprising a lock bar moveably attached to the second side of said frame via a bar attachment, said lock bar and attachment being adapted to hold said tension by pinching said elongate member at the second end between said bar and second frame side.

25. The device of claim 24, wherein said lock bar has a generally circular cross section and said bar attachment is adapted to constrain said bar from rotational movement.

26. The device of claim 24, wherein said bar attachment is adapted to mechanically constrain movement of said lock bar into the inside of said frame and away from said upper surface about the second side of the frame.

27. The device of claim 24, wherein when said lock bar and bar attachment fail to hold said tension, then said teeth of said comb are adapted to fully pierce and engage said elongate member at said second end and maintain said tension.

28. The device of claim 24, wherein said bar is attached inside the frame at the second side via a bar attachment that is adapted to mechanically constrain movement of said bar into the inside of said frame and away from said upper surface about the second side of frame.

29. The device of claim 24, wherein said lock bar is attached outside the second side of said frame via a bar attachment that is adapted to mechanically constrain movement of said lock bar into the inside of said frame and away from said upper surface about the second side of the frame.

30. The device of claim 19, and further comprising a needle attached to the second end of said elongate member.

31. The device of claim 30, wherein said frame has an opening sized to allow passage of said needle through it.

32. The device of claim 31 further comprising a needle attached to the second end of the elongate member wherein said lock bar is adapted to move away from said second frame side to a sufficient distance so as to allow the needle to pass around said lock bar.

33. The device of claim 32 adapted to be used in conjunction with a needle guide, wherein said needle guide comprises integral slots and is sized to removably fit, via said slots, over said lockbar in order to restrain said lock bar, and said needle guide is adapted to mitigate or prevent damage from said needle to said lock bar and bar attachment when said needle guide is placed over said lock bar.
